(19) United States
(12) Patent Application Publication
(10) Pub. No.: US 2006/0147508 A1
(43) Pub. Date: Jul. 6, 2006

(54) Concurrent Enhancement of Skin Penetration of Organic Base Active Agents and Organic Hydroxy Acid Active Agents as Their Ion-Pair Complexes

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 11/307,729

(22) Filed: Feb. 18, 2006

Related U.S. Application Data
(63) Continuation-in-part of application No. 10/265,000, filed on Oct. 4, 2002.
(63) Continuation-in-part of application No. 10/290,933, filed on Nov. 7, 2002.
(63) Continuation-in-part of application No. 10/280,519, filed on Oct. 25, 2002.
(63) Continuation-in-part of application No. 10/439,349, filed on May 15, 2003.
(63) Continuation-in-part of application No. 10/394,851, filed on Mar. 22, 2003, now abandoned.

(51) Int. Cl. A61K 31/66; A61K 31/665; A61K 31/51; A61K 31/455; A61K 31/4415

(52) U.S. Cl. 424/448; 514/101; 514/102; 514/350; 514/356; 514/276

(57) ABSTRACT

This invention relates to concurrent enhancement of skin penetration of certain organic nitrogen heterocyclic base active agents and certain organic acid active agents, especially organic hydroxy acids. This concurrent enhancement of skin penetration also provides enhanced bioavailability of both organic nitrogen heterocyclic base active agent and organic hydroxy acid active agent. The organic nitrogen heterocyclic base active agents and organic hydroxy acid active agents are first combined to form an ion-pair complex, which includes an in-situ mixing process, and said ion-pair complex is applied topically, whereupon said ion-pair complex undergoes enhanced skin penetration and upon reaching uppermost living part of skin and having reached therein the physiological pH of 7.4, said ion-pair complex dissociates to release its constituent organic nitrogen heterocyclic base active agent and said organic acid active agent in a bioavailable form.

This application is a continuation-in-part of U.S. patent application Ser. No. 10/265,000 filed Oct. 4, 2002. This application is also a continuation-in-part of U.S. patent application Ser. No. 10/280,519 filed Oct. 25, 2002. This application is also a continuation-in-part of U.S. patent application Ser. No. 10/290,933 filed Nov. 7, 2002. This application is also a continuation-in-part of U.S. patent application Ser. No. 10/394,851 filed Mar. 22, 2003. This application is also a continuation-in-part of U.S. patent application Ser. No. 10/439,349 filed May 15, 2003.

This invention relates to concurrent enhancement of skin penetration of certain organic nitrogen heterocyclic base active agents and certain organic acid active agents, especially organic hydroxy acids. This concurrent enhancement of skin penetration also provides enhanced bioavailability of both organic nitrogen heterocyclic base active agent and organic hydroxy acid active agent. The organic nitrogen heterocyclic base active agents and organic hydroxy acid active agents are first combined to form an ion-pair complex, which includes an in-situ mixing process, and said ion-pair complex is applied topically, whereupon said ion-pair complex undergoes enhanced skin penetration and upon reaching uppermost living part of skin and having reached therein the physiological pH of 7.4, said ion-pair complex dissociates to release its constituent organic nitrogen heterocyclic base active agent and said organic acid active agent in a bioavailable form.

The cosmetic and pharmaceutical benefits of certain organic base active agents and certain organic acid active agents in topical compositions are well known in the prior art. It is thus of great consumer importance to develop methods to either enhance or control (retard) the rate of topical penetration of such active agents. While certain organic base active agents may have a high pH incompatible with skin, the organic acid active agents may have a low pH incompatible with skin, as is well known in the prior art. It is not either surprising or unexpected that combinations of organic base active agents with certain acids, such as acid-base salts, acid-base ion-pairs, and acid-base amphoteric quaternium salts, which obviously have a more skin compatible pH profile, have been reported in the prior art as a means to either cause the skin penetration enhancement of an organic base active agent or an organic acid active agent. However, it is surprising that no prior art methods have disclosed concurrent skin penetration enhancement of both an organic base active agent and an organic acid active agent. While it is well known that human skin has a somewhat low pH profile, from about 5.0 to 6.0, and most typically about 5.5, it is worthy of mention that human skin also contains a relatively large amount of basic amino acids such as lysine and histidine in its protein structure. It is thus well known in the prior art that the application of an acid—base quaternium salt, such as a large number of commercially available quaternium skin and hair conditioning agents, are held on the upper layers of skin or hair, thus hindering their penetration into the deeper layers of skin. It is also well known in the prior art that the negative charge that is typically found on skin or hair binds with the positive charge of such quaternium conditioning agents. Similarly, when an acidic active agent is applied to skin it is known in the prior art to bind with free base (epsilon-amino) group of lysine or free base (imidazole heterocyclic) group of histidine, thus retarding further penetration of such acid active agents from upper layers to lower layer of skin.

While it is not unexpected that the pH profile of an ion-pair combination of an organic base active agent and an organic acid active agent should be desirable from skin compatibility point of view, the concurrent skin penetration in an enhanced mode of both the organic base active agent and the organic acid active agent should be highly desirable for cosmetic and topical pharmaceutical applications, especially when both said base and said acid active agents have a complementary profile of prior known desirable benefits.

The present invention relates to concurrent enhancement of skin penetration of organic base active agents and organic acid active agents, especially certain organic hydroxy acids. This concurrent enhancement of skin penetration also provides enhanced bioavailability of both organic base active agent and organic acid active agent, especially when organic base active agents and organic acid active agents are combined as the ion-pair complex, wherein one of the organic base active agents must be a nitrogen heterocyclic base, for example niacinamide, with a pH of more than 3.0 but less than 7.0 when said base is tested as one percent solution or suspension in water or a mixture of water and a water-miscible organic solvent. If the organic base active agent is not a nitrogen heterocyclic base, for example benzocaine, then the inclusion of an ion-pair complex of an organic nitrogen heterocyclic base and an organic acid active agent, even in catalytic amounts such as 0.1 percent by weight based on the total weight of the ion-pair complex of organic base active agent (but which is not an organic nitrogen heterocyclic base) and an organic acid active agent, results in concurrent enhanced penetration of both the said organic base active agent (but which is not an organic nitrogen heterocyclic base) and the organic acid active agent. If greater than catalytic amount of said ion-pair complex of an organic nitrogen heterocyclic base and an organic acid active agent is included in combination with an ion-pair complex of an organic base active agent (but which is not an organic nitrogen heterocyclic base) and an organic acid active agent, then concurrent skin penetration enhancement of all said active agents occurs; the organic base active agent (but which is not an organic nitrogen heterocyclic base), and the organic acid active agent that is bound by ion-pair bond with said organic base active agent, and the organic nitrogen heterocyclic base, and the organic acid active agent that is bound by ion-pair bond with said organic nitrogen heterocyclic base; and all of he above are thus released concurrently in enhanced amounts at the lower, living part of skin.

This invention also relates to an in-situ process for the preparation of ion-pair complexes of organic base active agents and hydroxy acids by the combination of an equimolar weight percent ratio of said organic base active agents and said hydroxy acids. The inclusion of a solubilizing agent such as water, alcohol, glycerin, glycols, polyethylene glycol, and N-methyl pyrrolidone is beneficial.

This invention also relates to a method of enhanced topical penetration of an ion-pair complex of an organic base active agent and an organic hydroxy acid active agent upon upper, non-living layers of skin, and comprising; (1) said ion-pair complex is applied topically on upper, non-living layers of skin, and whereupon said ion-pair complex undergoes enhanced skin penetration as said ion-pair complex chemical entity without any dissociation, and (2) upon reaching lower, living layers of skin, and having reached the physiological pH of 7.4, said ion-pair complex dissociates and releases its constituent organic base and organic hydroxy acid active agent moieties.

This invention also relates to a method of topical application of an ion-pair complex of an organic hydroxy acid active agent with an organic nitrogen heterocyclic base active agent, which comprises; (i) the in-situ generation of said ion-pair complex of an organic hydroxy acid active agent with an organic nitrogen heterocyclic base active agent by mixing of said Hydroxy Acid and said organic nitrogen heterocyclic base in a 1:1 molar weight percent ratio, and wherein said organic nitrogen heterocyclic base having a pH of lower than 7.0 but higher than 3.0, when tested as a 1 percent solution or suspension in distilled water, and (ii) a solubilizing agent is included in 10 to 90 percent by weight, based the combined weight of said organic nitrogen heterocyclic base and said organic hydroxy acid, and (iii) mixing temperature of from 20 degrees Celsius to 90 degrees Celsius, and (iv) the ion-pair complex thus generated with a solubilizing agent is applied topically.

This invention also relates to concurrent skin penetration enhancement of organic base active agents that includes niacinamide and niacin esters, and hydroxy acids that includes ascorbic acid, lactic acid, Mandelic acid, glycolic acid, Hydroxycitric acid, and salicylic acid, that are prepared by an in-situ process from the combination of organic base active agents such as niacinamide and pyridoxine with said organic acids.

A number of organic bases are known to be active agents for pharmaceutical and cosmetic applications. Vitamins, topical analgesics, alkaloids, and plant extracts are such examples. Niacin, also known as vitamin B.sub.3, is the common name for nicotinic acid. The physiologically active form of niacin is niacinamide, also a member of the vitamin B.sub.3 family of compounds. Niacin and niacinamide (nicotinic acid amide) function in the body as components of two coenzymes: nicotinamide adenine dinucleotide (NAD) and nicotinamide adenine dinucleotide phosphate (NADP). Until recently, these vitamin B.sub.3 compounds were used exclusively to treat niacin deficiency and pellagra. Today, however, vitamin B.sub.3 compounds have also found use in the area of skin care actives. The enhancement of skin penetration of vitamin B.sub.3 compounds by topical application is of high consumer interest. However, such enhancement of skin penetration has not been an easy task. For example, when applied to the skin in crystalline form (i.e., powder), vitamin B.sub.3 compounds tend to impart a rough feel to the skin. In the past, the crystalline vitamin B.sub.3 compounds were solubilized in a polar solvent before application to skin, thus alleviating the rough feel of the crystals. However, solubilization reduced the efficacy of the vitamin B.sub.3 compound upon contact with the skin. These problems have been discussed by Walling et al. (US Pat. No. 6,455,055) in detail. Walling et al. also reported the need for cosmetic compositions comprising unsolubilized crystalline vitamin B.sub.3 compound(s) and discovered certain cosmetic formulations incorporating the crystalline vitamin B.sub.3 compounds of specific particle size in combination with an emollient. However, such formulations were only applicable to lipstick delivery system, and hence of very limited applicability in other cosmetic products. It is also difficult to obtain niacinamide or niacin that meets the strict particle size specifications mentioned by Walling et al. SaNogueira et al. (U.S. Pat. No. 6,174,533) discuss that while a variety of compounds have been described in the art as being useful for regulating fine lines, wrinkles, acne, pimples, and other forms of undesirable skin surface texture, niacinamide and niacin have shown most promise in regulating skin conditions including fine lines, wrinkles, uneven or rough surface, and photo-damaged skin. Sauermann et al. (U.S. Pat. No. 6,428,779) report the need for antiaging compositions. According to these authors, skin ages as a result of endogenous, genetically determined influences. Exogenous factors, such as UV light and chemical noxae, can have a cumulative effect and accelerate the natural aging processes.

This results in numerous degenerative processes that lead, depending on the extent of the influencing factors, inter alia, to the following structural changes and damage in the dermis and epidermis (e.g. also to dermatoheliosis); a) Degeneration of the microvascular system; b) Flaccidity and development of wrinkles, partly due to a decrease in and crosslinking of collagen, accumulation of glycosaminoglycan (base substance) and solar elastosis (elastin clumping); c) Flattening of the retial cones. This is associated with the reduction in the area between the dermis and epidermis via which substances are exchanged for nutrition and purification of the epidermis; d) Restricted regenerative turnover in the epidermis, associated with defective development of the horny layer (disturbed hornification), leading to drying out of the skin, to roughness of the skin and to chapping of the skin; e) Defective regulation of cell division (proliferation) and cell maturation (differentiation) in the epidermis, which results in cellular atypia and atrophies and the loss in polarity; and f) Local hyper- and hypo pigmentation and abnormal pigmentation (age spots).

It would appear from the above biochemical problems related to skin disorders that high penetration and bioavailability of organic base active agents, such as niacinamide, pyridoxine, thiamine, or niacin esters from their topical applications is very important, but it is not easily achievable due to limited solubility and stability of said organic base active agents in various cosmetic compositions. It would thus be of great importance to enhance skin penetration of organic base active agents, such as niacinamide, pyridoxine, thiamine, or niacin esters that could circumvent the above problems of poor topical penetration. Furthermore, if skin penetration enhancement of organic base active agents, such as niacinamide, pyridoxine, thiamine, or niacin esters could be done by a method in combination with another agent that also has complementary topical beneficial properties, and wherein the enhancement of skin penetration of organic base active agents, such as niacinamide, pyridoxine, thiamine, or niacin esters and said another agent is concurrent, then such a method would be of even greater consumer interest.

Niacinamide, pyridoxine, thiamine, and niacin esters, as members of Vitamin B3 group, are well known for their various skin beneficial properties. The combination of organic base active agents, such as niacinamide, pyridoxine, thiamine, or niacin esters with other skin beneficial ingredients has been used in the prior art to develop combinations with synergistic skin beneficial properties. For example, Jacobson et al. (US Patent Application 20010049382 and 20020034482) describe niacin and certain synthetic esters of niacin that are useful for the treatment of hyperlipidemia and for improving the delivery of oxygen to cells. Horrobin et al. (U.S. Pat. No. 6,015,821) describe the preparation of esters of nicotinic acid that are useful for the control of dermatological disorders, among other applications. It would be advantageous if such esters can be made into enhanced bioavailable forms that can also offer synergistic efficacy for the alleviation of targeted skin ailments. Niacinamide and niacin have been used for several skin beneficial formulations. For example, a commercial product called “Papulex” is a very good selling acne treatment product that contains 4% niacinamide in a gel form marketed by Euroderma, Surrey, England. Zhang et al. (US Patent Application 20020106384) describe skin-whitening compositions that contain niacinamide. Robinson et al. (U.S. Pat. No. 6,444,647) describe a skin care composition that contains a combination of niacinamide and salicylic acid. However, this formulation must also contain farnesol and phytantriol as skin care actives for its efficacy. It is not clear if the skin beneficial effect is from the combination of niacinamide and farnesol or phytantriol, or a mixture of all the ingredients. Similarly, Bissett et al. (U.S. Pat. No. 6,183,761) describe a combination of niacinamide and salicylic acid for regulating skin disorders, but this composition must also include polycyclic compounds, some of which are anti-inflammatory in their property. It is thus not clear again if the efficacy of this formulation is actually from polycyclics, and not from niacinamide and salicylic acid. In the above cases, skin penetration enhancement of niacinamide, salicylic acid, or niacinamide salicylate was not reported. Fitzjarrell (U.S. Pat. No. 6,432,430) has prepared an exfoliating scrub that contains niacinamide for skin beneficial effects. However, this product also contains harsh scrubbing agents that expose fresh cells to permit the absorption of niacinamide into the skin. This is both inconvenient and sometimes painful or can cause skin irritation when a consumer with sensitive skin has to scrub skin. It would be much more advantageous if a niacinamide composition was more easily absorbed, more bioavailable, and did not require scrubbing the skin with an exfoliating product prior to the application of composition to enhance the skin penetration of niacinamide or other organic base active agents. Another acne treatment spray composition has been claimed by Fitzjarrell (U.S. Pat. No. 5,989,523), but for optimum benefits an exfoliating scrub is recommended prior to the application of the niacinamide spray composition. U.S. Pat. No. 5,968,528 (Deckner et al.) describes niacinamide compositions beneficial for skin compatibility. U.S. Pat. No. 5,962,482 (Bissett) describes cellulite reduction with a niacinamide composition. The oily appearance of skin can be controlled by a niacinamide composition described in U.S. Pat. Nos. 5,980,921 and 5,833,998 (Biederman et al.). U.S. Pat. Nos. 6,238,678 and 5,939,082 (Oblong et al.) describe a niacinamide composition for the treatment of skin aging. It would thus be obvious if methods for niacinamide compositions with enhanced absorption, better bioavailability, and synergistic skin care benefits could be developed that are easy for their topical application, stable, and cosmetically appealing.

A number of organic acids are also well known for their skin beneficial properties. U.S. Pat. No. 5,861,432 (Sklar) describes the use of glycolic acid in an acne treatment formulation. Glycolic acid has been used in many cosmetic formulations for improved skin appearance. There are two main theories on how glycolic acid works. The first theory proposes that the glycolic acid produces a mild sub clinical irritation which stimulates the epidermis to produce fresh skin, while the second theory proposes that glycolic acid weakens the intercellular bonding of the corneocytes in a manner similar to both water and retinoids. Unfortunately, little objective data regarding the effectiveness of alpha-hydroxy acid (AHA), such as glycolic acid, has been published thereby leaving the industry to rely on anecdotal information, which is difficult to quantify. It is quite clear that many of the topical cosmetics incorporating glycolic acid or other alpha-hydroxy acids have insufficient concentrations to accomplish their objectives. The human skin is comprised of two principal components, the avascular epidermis and the underlying vascular dermis. The epidermis consists of four layers: the stratum corneum, stratum granulosum, stratum spinosum and stratum basale. The dermis mainly consists of collagen, elastin fibers and ground substances including glycosaminoglycan. There are two forms of skin aging: intrinsic aging, also known as chronological aging and extrinsic aging, also known as photo aging. The aging process normally involves the dermis.

Intrinsic aging is a degenerative process attributed to declining physiologic functions and capacities. Extrinsic aging is caused by external factors such as sunlight, radiation, and air pollution. Alpha-hydroxy acids (AHA's) have been used topically in the prior art on keratinization (epidermal layer) where the effects are clinically detectable by the formation of a new stratum corneum. AHA's also have dermal effects. Topical applications of AHA's have caused increased amounts of mucopolysaccharides and collagen and increased skin thickness without detectable inflammation. The benefits of the AHA have caused them to be incorporated into cosmetic products for purposes such as cleansing, conditioning, dry skin etc. AHA's are categorized as nontoxic and have been used as skin desquamative agents, especially in routine use for acne, wrinkles, photo aged skin and pigmented disorders. Mandelic acid, another AHA, has been claimed by Yu et al. (U.S. Pat. Nos. 5,677,339 and 5,654,336) in a topical composition for skin wrinkles reduction. Glycolic and lactic acids have been claimed in pimples and skin redness reduction compositions by Slavtcheff et al. (U.S. Pat. Nos. 5,614,201 and 5,482,710). Alliger (U.S. Pat. No. 5,516,799) describe the use of glycolic acid for treating small mouth ulcers. Shaffer et al. (U.S. Pat. No. 5,760,079) describe hydroxy acids for treating striae distensae (stretch marks). Perricone (U.S. Pat. No. 6,417,226) has claimed Hydroxytetronic acid in a skin whitening composition. Other AHA's have shown skin-whitening effects, as mentioned by Zhang et al. (US Patent Application 20020106384).

There is no doubt that alpha hydroxy acids, alpha ketoacids and related compounds are therapeutically effective for topical treatment of various cosmetic conditions and dermatological disorders including dry skin, acne, dandruff, keratoses, age spots, wrinkles and disturbed keratinization. However, the compositions containing these acids may irritate human skin on repeated topical applications due to lower pH of the formulations, as is well known in the prior art. The irritation may range from a sensation of tingling, itching and burning to clinical signs of redness and peeling. Causes for such irritation may arise from the following: Upper layers of normal skin have a pH of 4.2 to 5.6, but the compositions containing most alpha hydroxy acids or alpha ketoacids have pH values of less than 3.0. For example, a topical formulation containing 7.6% (1 M) glycolic acid has a pH of 1.9, and a composition containing 9% (1 M) lactic acid has the same pH of 1.9. These compositions of lower pH on repeated topical applications can cause a drastic pH decrease in the stratum corneum of human skin, and provoke disturbances in intercorneocyte bondings resulting in adverse skin reactions, especially to some individuals with sensitive skin. Moreover, with today's state of the art it is still very difficult to formulate a lotion, cream or ointment emulsion which contains a free acid form of the alpha hydroxyacid, and which is physically stable as a commercial product for cosmetic or pharmaceutical use.

When an AHA or alpha-ketoacid is reacted in equimolar or equinormal amounts with a metallic alkali such as sodium hydroxide or potassium hydroxide to form the corresponding salt derivative, said AHA becomes therapeutically ineffective for many topical applications, as is well known in the prior art. The reasons for such loss of therapeutic effects are believed to be as follows: The intact skin of humans is a very effective barrier to many natural and synthetic substances. Cosmetic and pharmaceutical agents may be pharmacologically effective by oral or other systematic administration, but many of them are much less or totally ineffective on topical application to the skin. Topical effectiveness of a pharmaceutical agent depends on two major factors, (a) bioavailability of the active ingredient in the topical preparation and (b) percutaneous absorption, penetration and distribution of the active ingredient to the target site in the skin. For example, a topical preparation containing 5% salicylic acid is therapeutically effective as a keratolytic, but that containing 5% sodium salicylate is not an effective keratolytic composition. The reason for such difference is that salicylic acid is in bioavailable form and can penetrate the stratum corneum, but sodium salicylate is not in bioavailable form for this specific skin beneficial function and cannot penetrate the stratum corneum of the skin. In the case of alpha hydroxy acids, a topical preparation containing 5% glycolic acid is therapeutically effective for dry skin, but that containing 5% sodium glycolate is not effective. The same is true in case of 5% lactic acid versus 5% sodium lactate. The reason for such difference is that both glycolic acid and lactic acid are in bioavailable forms and can readily penetrate the stratum corneum, but sodium glycolate and sodium lactate are not in bioavailable forms for the intended specific skin beneficial functions and cannot penetrate the stratum corneum of the skin. When an alpha hydroxy acid or alpha ketoacid is reacted in equimolar or equinormal amounts with ammonium hydroxide or an organic base of smaller molecule the salt derivative thus formed shows greatly diminished therapeutic effects for certain cosmetic conditions such as dry skin, but otherwise it has lost most of its potency for other dermatological disorders such as wrinkles, keratoses, age spots and skin changes associated with aging. Thus, the salt formation of organic hydroxy acids with a base is generally known to cause poor skin penetration of said hydroxy acids.

It would thus be obvious to combine the benefits of organic base active agents, such as niacinamide, and organic hydroxy acids in a single composition. It would also be obvious to enhance the skin penetration of niacinamide and hydroxy acid in said combination. It would also be obvious to enhance skin penetration of an organic base active agent, such as niacinamide, and hydroxy acid in said combination concurrently, i.e., the skin penetration of niacinamide and hydroxy acid is enhanced simultaneously. However, the latter has not been disclosed in the prior art. For example, U.S. Pat. No. 5,681,554 (Cannell et al.) discloses the benefits of Niacinamide Ascorbate (col. 7, line 27-38); the enhanced skin or hair penetration of which is not disclosed. The present invention provides a practical solution to this problem.

U.S. Pat. No. 4,888,354 (Chang et al.) discloses skin penetration enhancement of certain organic free bases in combination with acid addition salts of said free bases. This disclosure is of great interest, as the skin penetration enhancement of only the free base is achieved only when it is also in combination with its addition salt with an acid such as lactic acid. The skin penetration enhancement is not achieved either with the free base alone, or with the acid addition salt of free base alone. It should be noted that acid addition salt of base does not have any better skin penetration than the free base itself. It is only a combination of both the free base and its acid addition salt that provides enhanced skin penetration, according to Chang et al.

JP 361122225A (Abe et al.) discloses a method to increase the transcutaneous permeability and absorptivity of drug active agents such as local anesthetic, antihistaminic, and antibiotics when such agents are used in combination with a nicotinic acid ester. The absorptivity of said drug agents can be further in combination with a polar agent, such as alcohol, glycerol, and lactic acid. It is not known if the transcutaneous permeability and absorptivity of both nicotinic ester and lactic acid is also increased concurrently. Abe et al. only disclose an increase of the transcutaneous permeability and absorptivity of drug active agents such as local anesthetic, antihistaminic, and antibiotics, and not the combination of nicotinic ester and lactic acid.

JP 63132838A (Kao) discloses a method to increase the transcutaneous permeability and absorptivity of drug active agents such as corticosteroids when such agents are used in combination with a nicotinic acid ester and/or salicylic acid ester (not salicylic acid, but salicylic acid ester). The absorptivity of said drug agents can be further in combination with a polar agent, such as dimethylsulfoxide, propylene glycol and triglycerin monostearate. It is not known if the transcutaneous permeability and absorptivity of both nicotinic ester and lactic acid is also increased concurrently. JP 63132838A only discloses an increase of the transcutaneous permeability and absorptivity of drug active agents such as corticosteroids.

EP 000325969A1 (Brenner) discloses a combination of methylxanthine, a nicotinic ester, and, optionally, salicylic acid for the treatment of alopecia. Brenner does not teach concurrent skin penetration enhancement of both nicotinic ester and salicylic acid.

Chang, Abe, Kao, and Brenner do not disclose any concurrent enhancement of topical penetration of two or more beneficial agents that are an ion-pair combination of an organic base active agent and an organic hydroxy acid, and wherein said ion-pair combination converts back into their original organic base active agent and organic hydroxy acid components upon absorption into the deeper layers of skin and having reached the physiological pH of 7.4 therein, as in Equation 1.
Niacinamide Salicylate (skin absorption) (physiological pH of 7.4)=Niacinamide+Salicylic acid (Equation 1).

U.S. patent application Ser. No. 20050059644 (Rood et al.) discloses certain dermatological compositions that contain a combination of both a hydroxy acid and its salt. Such compositions may also contain additional agents. However, Rood et al. do not disclose any method or composition for increasing the topical penetration of said hydroxy acid or its salt, especially in combination with another active agent wherein the topical penetration of both the hydroxy acid and the additional agent are increased concurrently.

U.S. Pat. No. 6,429,218 (Scivoletto) discloses the enhancement of the efficacy of niacin in a topical composition by including other active agents, such as glycolic acid and salicylic acid. Scivoletto does not disclose concurrent enhancement of skin penetration of both niacin and other active agents such as glycolic acid and salicylic acid used concurrently. Scivoletto teaches a method of only controlling niacin in topical compositions.

U.S. Pat. No. 5,939,082 (Oblong et al.) discloses a combination of two active agents, niacinamide, an organic base, and salicylic acid, an organic acid, for example, which are useful for certain skin conditions. However, Oblong et al. do not disclose any skin penetration enhancement of either the organic base or the organic acid, or concurrently of both.

U.S. Pat. No. 5,690,967 (Yu et al.) discloses improved topical delivery of lactic acid with certain amphoteric agents, when the pH of said composition is 4.2 or less. Yu et al. do not disclose the concurrent improved topical delivery of amphoteric agents that are used in combination with lactic acid.

U.S. Pat. No. 5,681,853 (Yu et al.) discloses improved topical delivery of hydroxy acids with certain amphoteric agents, when the pH of said composition is 4.2 or less. Yu et al. do not disclose the concurrent improved topical delivery of amphoteric agents that are used in combination with hydroxy acid.

U.S. Pat. No. 5,091,171 (Yu et al.) discloses improved topical delivery of hydroxy acid and polymeric hydroxy acid with certain amphoteric agents, when the pH of said composition is 4.2 or less. Yu et al. do not disclose the concurrent improved topical delivery of amphoteric agents that are used in combination with said hydroxy acid and polymeric hydroxy acid.

U.S. Pat. No. 5,783,601 (Tanahashi et al.) discloses certain salts of hydroxy acid with alkali metals, amines, and amphoteric agents for treating skin condition. Tanahashi et al. do not disclose the skin penetration enhancement of said salts of hydroxy acids.

U.S. Pat. No. 6,677,361 (Jacobson et al.) discloses certain chemically altered forms of niacin for topical applications. Jacobson et al. do not teach any method for the enhancement of topical penetration of such compositions.

The present invention discloses a method for concurrent enhancement of topical penetration of both an organic nitrogen heterocyclic base active agent and an organic hydroxy acid active agent. This method comprises the following steps. (1) The organic nitrogen heterocyclic base active agent and organic hydroxy acid active agent are combined in their equimolar weight percent amount to form an ion-pair complex. (2) The ion-pair complex is applied topically, whereupon said complex undergoes enhanced skin penetration, and the ion-pair penetrates the non-living layers of the uppermost part of skin intact, that is, the ion-pair penetrates this non-living part of skin as the ion-pair chemical entity. (3) The ion-pair chemical entity upon reaching the uppermost living part of skin located just below the non-living part of skin, and having reached therein the physiological pH of 7.4, said ion-pair complex dissociates and original constituent organic nitrogen heterocyclic base active agent and organic acid active agent are released in that part of skin. The organic nitrogen heterocyclic base active agent and organic acid active agent continue their further penetration into skin. It should be noted at this point that both the total amount and the speed of penetration of said ion-pair is at least 50% greater than both the total amount and the speed of organic nitrogen heterocyclic base active agent and organic acid active agent applied topically as independent chemical entities, that is, not as their ion-pair complex.

The organic nitrogen heterocyclic base active agent is selected from a large number of such bases that have a pH of greater than 3.0 but less than 7.0, when said base is tested as a 1 per cent solution or suspension in water. The examples include purines, pyrimidines, nucleic acids, nucleotides, oxazoles, pyrroles, pyrazoles, imidazoles, pyridines, quinolines, quinazolines, quinoxalines, vincamine, vinpocetin, alkaloids, and combinations thereof. The examples are so numerous that FIG. 1 shows just few such five member nitrogen heterocyclic bases. There are six member nitrogen heterocyclic bases, and fused ring bases. The organic nitrogen heterocyclic base active agents of highest importance are selected from niacinamide, thiamine, pyridoxine, yohimbine, vincamine, vinpocetin, and quinine.

[FIG. 1].

The organic hydroxy acid active agent is selected from a large number of such hydroxy acids available, which includes Glycolic Acid, Malic Acid, Lactic Acid, Mandelic Acid, Ascorbic Acid, Phytic Acid, Salicylic Acid, Aleuritic Acid, Tartaric Acid, Citric Acid, Hydroxytetronic Acid, Glucuronic Acid, Hyaluronic Acid, Mucic Acid, Galacturonic Acid, Gluconic Acid, Saccharic Acid, Glucoheptonic Acid, alpha-Hydroxybutyric Acid, Tartronic Acid, alpha-Hydroxyisobutyric Acid, Isocitric Acid, alpha-Hydroxyisocaproic Acid, Dihydroxymaleic Acid, alpha-Hydroxyisovaleric Acid, Dihydroxytartaric Acid, beta-Hydroxybutyric Acid, Dihydroxyfumaric Acid, beta-Phenyllactic Acid, Atrolactic Acid, Galactonic Acid, Pantoic Acid, Glyceric Acid, and their derivatives, and combinations thereof.

Furthermore, the preparation of said ion-pair is achieved by an in-situ process, wherein the organic nitrogen heterocyclic base active agent and hydroxy acid active agent are combined in an equimolar weight percent ratio in the presence of a solubilizing agent, such as water, alcohol, glycol, polyglycol, and glycol ether, to facilitate said ion-pair complex formation. The ion-pair combinations formed by the in-situ process are independent chemical species, i.e., they are not mere physical mixtures of organic base active agent and hydroxy acid. However, upon application to skin and their rapid absorption into the deeper layers of skin said ion-pair chemical species are converted back into their original organic nitrogen heterocyclic base active agent and organic hydroxy acid active agent, the absorption of both organic base active agent and hydroxy acid having occurred at much increased rate and amount than when the organic base active agent or the hydroxy acid topically are applied alone as individual chemical entities.

It is both unexpected and surprising that the inclusion of even a catalytic amount, for example 0.1 weight percent of an ion-pair complex of an organic nitrogen heterocyclic base and organic hydroxy acid, the skin penetration of certain organic free bases in combination with acid addition salts of said free base, as disclosed by Chang et al. (U.S. Pat. No. 4,888,354), can be achieved by the present invention. This finding is of great interest, as the skin penetration enhancement of only the free base is achieved only when it is also in combination with its addition salt with an acid such as lactic acid, according to Chang et al. The skin penetration enhancement is not achieved either with the free base alone, or with the acid addition salt of free base alone. It should be noted that acid addition salt of base does not have any better skin penetration than the free base itself, according to Chang et al. It is only a combination of both the free base and its acid addition salt that provides enhanced skin penetration, according to Chang et al. The inclusion of the free base is not necessary for the skin penetration enhancement of free base in combination with its addition salt with an acid such as lactic acid when a catalytic amount of an ion-pair complex of an organic nitrogen heterocyclic base with said acid is included. This is further illustrated in FIG. 2, wherein the skin penetration of certain active ingredients reported by Chang et al. (U.S. Pat. No. 4,888,354; column 9, line 31, Table I) was repeated by the teachings of the present invention and with the inclusion of niacinamide lactate ion-pair complex in a catalytic amount of 0.1% by weight, based on the combined weight of base and its acid addition salt in Table I of Chang et al.

[FIG. 2].

The present invention also discloses a method of topical application of an ion-pair complex of an organic hydroxy acid active agent with an organic nitrogen heterocyclic base active agent. This method of topical application of said ion-pair complex results in enhanced absorption of said ion-pair complex through the upper, non-living layers of skin. This method comprises the following steps. (1) Said ion-pair complex of an organic hydroxy acid active agent with an organic nitrogen heterocyclic base active agent is first generated by mixing of said Hydroxy Acid and said organic nitrogen heterocyclic base in a 1:1 molar weight percent ratio, and wherein said organic nitrogen heterocyclic base having a pH of lower than 7.0 but higher than 3.0, when tested as a 1 percent solution or suspension in distilled water. (2) A solubilizing agent is included in 10 to 90 percent by weight, based the combined weight of said organic nitrogen heterocyclic base and said organic hydroxy acid. The solubilizing agent is selected from water, alcohol, glycol, polyglycol, glycol ether, and combinations of said solubilizing agents. (3) Mixing temperature of (1) and (2) is from 20 degrees Celsius to 90 degrees Celsius. (4) And, finally, said ion-pair complex is applied topically. The ion-pair complex of an organic hydroxy acid active agent with an organic nitrogen heterocyclic base active agent is selected from niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxy citrate, pyridoxine aleurate, pyridoxine salicylate, pyridoxine hyaluronate, thiamine lactate, thiamine glycolate, thiamine malate, thiamine mandelate, thiamine ascorbate, thiamine phytate, thiamine citrate, thiamine hydroxy citrate, thiamine aleurate, thiamine salicylate, thiamine Hyaluronate, and combinations thereof.

The present invention also discloses a method of enhanced topical penetration of an ion-pair complex of an organic nitrogen heterocyclic base active agent and an organic hydroxy acid active agent upon upper, non-living layers of skin. This is achieved in the following steps. (1) Said ion-pair complex is applied topically on upper, non-living layers of skin, and whereupon said ion-pair complex undergoes enhanced skin penetration as said ion-pair complex chemical entity without any dissociation. (2) Upon reaching lower, living layers of skin, and having reached therein the physiological pH of 7.4, said ion-pair complex dissociates and releases its constituent organic nitrogen heterocyclic base and organic hydroxy acid active agent moieties. (3) A solubilizing agent, and/or a cosmetically or pharmaceutically acceptable topical base may also be included.

The ion-pair complexes of an organic nitrogen heterocyclic base active agent and an organic hydroxy acid active agent are preferably made by an in-situ process in the present invention. Equation 2 shows the preparation of the ion-pair compound, niacinamide lactate, by mixing an equimolar weight percent of niacinamide, an organic nitrogen heterocyclic base active agent, and lactic acid, an organic acid active agent.
Niacinamide+Lactic Acid=Niacinamide Lactate (Equation 2).

The topical application of niacinamide salicylate results in rapid penetration, and both niacinamide and lactic acid are released in significantly increased amounts into deeper layers of skin.

Equation 3 shows the preparation of the ion-pair compound, niacinamide ascorbate, by mixing an equimolar weight percent of niacinamide, an organic base with prior known skin whitening benefits, and a prior known skin whitening agent, ascorbic acid, a hydroxy acid.
Niacinamide+Ascorbic Acid=Niacinamide Ascorbate (Equation 3).

The topical application of niacinamide Ascorbate results in rapid penetration, and both niacinamide and ascorbic acid are released in significantly increased amounts into deeper layers of skin upon the penetration of niacinamide Ascorbate therein.

Similarly, by mixing benzocaine with Salicylic acid in equimolar weight percent amounts in water solution, benzocaine salicylate is produced in-situ, as illustrated in Equation 4.
Benzocaine+Salicylic Acid=Benzocaine Salicylate (Equation 4).

The topical application of Benzocaine salicylate results in rapid penetration, and both benzocaine and salicylic acid are released in significantly increased amounts into deeper layers of skin upon the penetration of Benzocaine salicylate therein and having reached the physiological pH of 7.4 Both Benzocaine and salicylic acid are well known topical analgesic agents.

There are alternate methods for the preparation of ion-pair compounds of the present invention, for example, by mixing an inorganic acid salt of an organic base active agent such as yohimbine hydrochloride, with a metal salt of hydroxy acid, such as sodium Glycolate, as depicted in Equation 5. Both yohimbine and glycolic acid are well known skin brightening agents from the prior art.
Yohimbine hydrochloride+Sodium Glycolate=Yohimbine Glycolate+Sodium Chloride (Equation 5).

Multi-component ion-pair compounds of organic base active agents with hydroxy acids can also be made by the in-situ method by mixing the reacting components in proportionate equimolar weight percent quantities, as illustrated in Equation 6. The addition of water or other solubilizing agent is usually beneficial during the preparation of such ion-pair compounds, the amount of which can be optimized by solubility determination.
Niacinamide+Glycolic Acid+Hydroxycitric Acid+Ascorbic Acid+Salicylic Acid=Niacinamide Glycolate+Niacinamide Hydroxycitrate+Niacinamide Ascorbate+Niacinamide Salicylate (Equation 6).

The compositions in Equation 2 to 6 can also be made in anhydrous systems by using appropriate water-miscible organic solvent in the in-situ method disclosed in the present invention. The examples of water-miscible organic solvents include glycerin, propylene glycol, butylene glycol, polyethylene glycol, polypropylene glycol, methyl pyrrolidone, pyrrolidone, butylene glycol, hexylene glycol, methylpropanediol, glycol ethers, ethanol, methyl lactate, Triethyl citrate, isopropanol, and such.

The amount of organic nitrogen heterocyclic base active agent, such as niacinamide, niacin esters, yohimbine, or quinine, is from about 0.1% to about 50% by weight in the ion-pair complex, preferably from 5% to 20% by weight, most preferably from 1% to 10% by weight. The hydroxy acid is included in equimolar weight percent amount, based on the weight of organic base active agent in the ion-pair complex.

The amount of the solubilizing agent in the composition is from about 0% to about 90%. This is because the methods of topical application for the ion-pair complexes of an organic base active agent and a hydroxy acid can also include traditional water and oil emulsions, suspensions, colloids, solutions, or anhydrous systems. For anhydrous systems, the water is typically much less than 1%. The present invention thus permits the enhanced topical penetration of a wide variety of ion-pair complexes that can contain water or be anhydrous systems. Anhydrous systems may be preferred for certain applications, such as the preparation of high potency facial serums and skin whitening lotions, whereas water and oil emulsions and suspensions are typically preferred for lotion, cream, gel, paste, and such, as shall become clearer in the Examples section of this invention. It should be noted that said compositions are not claimed in the present invention.

The methods of enhanced topical delivery of the present invention can also utilize a variety of prior known delivery systems. The delivery system can comprise a base for lotion, cream, shampoo, serum, gel, salve, paste, spray, collodion, and such. The delivery system can be composed of one or more ingredients to provide skin elegance, skin feel, and enhanced bioavailability attributes popularly desired by the consumers.

The preferred pH of the ion-pair complexes is from about 3.0 to about 7.5, preferably from about 3.5 to about 5.5. The preferable pH is determined by the optimum stability of the ion-pair complex that is derived from the combination of an organic base active agent and a hydroxy acid. For example, FIG. 3 summarizes the pH profile of individual active agents and their ion-pair complexes. In FIG. 3, pH of ion-pair complexes in column 4 was determined from combining, in equimolar amounts, niacinamide in column 1, with appropriate organic acid in column 2, to give desired niacinamide derivative in column 3. This preparation was done in-situ in a deionized water solution by mixing 0.01 mole of niacinamide in 50 grams of deionized water and then determining the pH of the resulting solution, them preparing a solution or suspension of 0.01 mole of an organic acid in column 2 in 50 grams of deionized water and determining its pH. The solution of niacinamide in water obtained in column 1 is then combined with the solution or suspension of organic acid in water obtained in column 2, to provide a solution of niacinamide derivative of organic acid in deionized water as per column 3. The pH of niacinamide derivative thus obtained in column 3 is also indicated in column 3. The pH in column 3 was determined to be optimal for any compositions that also contained the ion-pair complexes in column 3. The teachings of the present invention are not limited to hydroxy acid, as further shown in an example of niacinamide lipoate in column 3.

[FIG. 3].

Additional skin, hair, and body beneficial ingredients, such as other anti-aging ingredients, vitamins, hormones, analgesics, anesthetics, sun screens, skin whiteners, anti-acne agents, anti-bacterial agents, anti-fungal agents, botanical extracts, pharmaceuticals, processing-aids, minerals, plant extracts, concentrates of plant extracts, emollients, moisturizers, skin protectants, humectants, silicones, skin soothing ingredients, colorants, perfumes, and like can be included with the ion-pair complexes for their topical penetration in accordance with the methods of the present invention.

The in-situ process of present invention is useful for the preparation of ion-pair complexes of organic base active agents and hydroxy acid. Such examples of ion-pair complexes include niacinamide salicylate, niacinamide lipoate, niacinamide mandelate, niacinamide lactate, niacinamide glycolate, niacinamide malate, niacinamide adenosine phosphate, niacinamide adenosine triphosphate, niacinamide ascorbate, niacinamide folate, niacinamide hydroxycitrate, niacinamide hydroxytetronate, niacinamide pantothenate, niacin salicylate, niacin lipoate, niacin mandelate, niacin lactate, niacin glycolate, niacin malate, niacin adenosine phosphate, niacin adenosine triphosphate, niacin ascorbate, niacin folate, niacin hydroxycitrate, niacin pantothenate, niacin hydroxytetronate, benzyl nicotinate lipoate (benzyl niacin lipoate), methyl nicotinate lipoate (methyl niacin lipoate), benzyl niacin ascorbate, methyl niacin ascorbate, benzyl niacin salicylate, methyl niacin salicylate, benzyl niacin pantothenate, methyl niacin pantothenate, benzyl niacin lactate, methyl niacin lactate, benzyl niacin malate, methyl niacin malate, lauryl niacin lipoate, lauryl niacin ascorbate, lauryl niacin salicylate, lauryl niacin lactate, methyl niacin glycyrrhetinate, niacinamide glycyrrhetinate, niacinamide glycyrrhizinate, niacinamide hyaluronate, niacinamide pyrrolidone carboxylate, benzyl niacin hyaluronate, benzyl niacin pyrrolidone carboxylate, niacinamide hydroquinone carboxylate, niacin hydroquinone carboxylate, methyl niacin hydroquinone carboxylate, benzyl niacin hydroquinone carboxylate, lauryl niacin hydroquinone carboxylate, methyl niacin ursolate, lauryl niacin ursolate, benzyl niacin ursolate, niacinamide ellagate, niacinamide rosmarinate, niacinamide chloroginate, methyl niacin ellagate, methyl niacin chloroginate, lauryl ellagate, lauryl chloroginate, lauryl rosmarinate, and methyl niacin rosmarinate.

### EXAMPLES

The following examples are presented to illustrate presently preferred practice thereof. As illustrations they are not intended to limit the scope of the invention.

All amounts are in weight percent.

### Example 1

In-Situ Process for Niacinamide Ascorbate. Column 1 describes the ingredients as they are used in the formulation. Column 2 describes the final percent weight resulting from the in-situ formation of ion-pair complex of niacinamide ascorbate from niacinamide and ascorbic acid.

| Ingredient | Column 1 | Column 2 |
|---|---|---|
| Glycerin | 69.1 | 69.1 |
| Ascorbic Acid | 9.0 | 0.0 |
| Niacinamide | 6.0 | 0.0 |
| Niacinamide Ascorbate | 0.0 | 15.0 |
| Deionized Water | 15.0 | 15.0 |
| Preservative | 0.9 | 0.9 |

Procedure: Mix all ingredients in a tank. A clear yellow solution is obtained.

### Example 2

Skin Penetration of Niacinamide, Ascorbic Acid, and In-Situ Generated Niacinamide Ascorbate (from Example 1).

| Ingredient | % Penetration |
|---|---|
| Niacinamide | 20.0 |
| Ascorbic Acid | 25.0 |
| Niacinamide Ascorbate | 72.0 |

Procedure. A 0.1 molar solution of ingredients in a mixture of glycerin and water, as shown in Example 1, was applied on a synthetic membrane, which was placed over a Franz Diffusion Cell. The ingredients migrating to the phosphate buffer part of diffusion cell were quantified. % Penetration is based on the amount of ingredient applied to synthetic membrane.

### Example 3

In-situ Preparation of Niacinamide Ascorbate in Combination with a Cosmetic Cleanser Base. Column 1 describes the ingredients as they are used in the formulation. Column 2 describes the final percent weight resulting from the in-situ formation of ion-pair complex of niacinamide ascorbate from niacinamide and ascorbic acid.

| Ingredient | Column 1 | Column 2 |
|---|---|---|
| Glycerin | 43.3 | 43.3 |
| Methyl paraben | 0.2 | 0.2 |
| Ascorbic Acid | 9.0 | 0.0 |
| Niacinamide | 6.0 | 0.0 |
| Niacinamide Ascorbate | 0.0 | 15.0 |
| Deionized Water | 15.0 | 15.0 |
| Phenoxyethanol | 0.9 | 0.9 |
| Na Cocoyl Isethionate | 20.0 | 20.0 |
| Na Methyl Cocoyl Taurate | 5.0 | 5.0 |
| Botanical Extract | 0.1 | 0.1 |
| Fragrance | 0.5 | 0.5 |

Procedure: Mix deionized water, ascorbic acid, and niacinamide in a tank. A clear solution is obtained. All of the other ingredients are then added, and the mixture is heated and stirred at 60 to 70 degrees C. for about five to ten minutes until the mixture is homogenous. The homogeneous mixture is cooled to room temperature. A paste-like product is formed. The stabilized niacinamide ascorbate composition is applied as a facial, hair, and body cleanser. It should be noted that when the composition is first mixed, as shown in Column 1, it is white in color. After preparation of the batch is complete, the product turns bright yellow, indicating the formation of niacinamide ascorbate, which is naturally yellow in color. The color meter readings were L 91.94, a -7.21, b 22.20. The application of this composition to skin results in rapid penetration of the ion-pair complex, Niacinamide Ascorbate, and the release of niacinamide and ascorbic acid into deeper layers of skin.

### Example 4

Stability Testing of Ion-Pair Complex of Example 3.

The paste of Example 3 was stored at room temperature in a sealed container in the presence of air. After six months the paste is still yellow. A calorimetric reading with a color meter, such as Hunter Color Meter, shows that the color reading has changed by only 5%, and the product is still stable, and has not separated into solid and liquid phases.

The color meter readings were L 91.43, a—7.03, b 24.46.

### Example 5

Rapid Penetration Niacinamide Ascorbate Ion-pair Complex in a Facial Gel Base.

| Ingredient | Column 1 | Column 2 |
|---|---|---|
| PEG-6 | 46.5 | 46.5 |
| NH4 Acryloylmethyltaurate | 1.0 | 1.0 |
| Ascorbic Acid | 9.0 | 0.0 |
| Niacinamide | 6.0 | 0.0 |
| Niacinamide Ascorbate | 0.0 | 15.0 |
| Deionized Water | 20.0 | 20.0 |
| Glycerin | 5.0 | 5.0 |
| Preservative | 0.5 | 0.5 |
| Vitamin E | 2.0 | 2.0 |
| Dimethicone | 4.0 | 4.0 |
| Dimethiconol | 4.0 | 4.0 |
| Cetyl Dimethiconol | 2.0 | 2.0 |

Procedure: All ingredients in Column 1 were mixed and heated at 40 to 50 C. for 30 minutes. The mixture was cooled to room temperature. A clear gel was obtained, with analysis reported in Column 2.

### Example 6

The In-Situ Preparation of 32.8% High Potency Niacinamide Lipoate from Niacinamide and Lipoic Acid in a Cosmetic Serum Base.

| Ingredients | Column 1 | Column 2 |
|---|---|---|
| Lipoic Acid | 20.6 | 0.0 |
| Niacinamide | 12.2 | 0.0 |
| Water | 33.2 | 33.2 |
| Propylene Glycol | 34.0 | 34.0 |
| Niacinamide Lipoate | 0.0 | 32.8 |

Procedure: All ingredients in Column 1 were mixed and heated at 40 to 50 C. for 30 minutes. The product was cooled. A thin solution of composition in Column 2 was obtained.

### Example 7

The In-Situ Process of a 42.9% high potency anti-aging and skin whitening Niacinamide Glutathione composition.

| Ingredients | Column 1 | Column 2 |
|---|---|---|
| Glutathione | 30.7 | 0.0 |
| Niacinamide | 12.2 | 0.0 |
| Deionized Water | 50.0 | 50.0 |
| Methylpropanediol | 7.1 | 7.1 |
| Niacinamide Glutathione | 0.0 | 42.9 |

Procedure: All ingredients in Column 1 were mixed. A solution of Niacinamide Glutathione was obtained, as shown in Column 2.

### Example 8

In-Situ Process of Niacinamide Salicylate and Niacinamide Ascorbate in a Cosmetic Gel Base.

| Ingredient | Column 1 | Column 2 |
|---|---|---|
| PEG-6 | 56.02 | 56.02 |
| Aristoflex AVC | 1.0 | 1.0 |
| Glycerin | 5.0 | 5.0 |
| Water | 20.0 | 20.0 |
| Preservative | 0.5 | 0.5 |
| Vitamin E | 0.5 | 0.5 |
| Niacinamide | 2.44 | 0.0 |
| Ascorbic Acid | 1.76 | 0.0 |
| Salicylic Acid | 1.38 | 0.0 |
| Dimethicone | 4.0 | 4.0 |
| Dimethiconol | 4.0 | 4.0 |
| Cetyl Dimethiconol | 2.0 | 2.0 |
| Galanga Ext. | 0.2 | 0.2 |
| Esculin | 0.5 | 0.5 |
| Boswellia Serrata Ext. | 0.2 | 0.2 |
| Methylsulfonylmethane | 0.5 | 0.5 |
| Niacinamide Ascorbate | 0.0 | 2.98 |
| Niacinamide Salicylate | 0.0 | 2.6 |

Procedure: Mix all ingredients in Column 1 and heat at 60 to 70 C. for 30 minutes. Cool to room temperature, and adjust pH to 4.5 with sodium hydroxide solution. A clear pale yellow gel of composition in Column 2 was obtained.

### Example 9

In-Situ Process of a Mixture of Niacinamide Lactate, Niacinamide N-Acetyl-Cysteinate, Niacinamide Ascorbate, Niacinamide Salicylate, and Niacinamide Lipoate in a Cosmetic Gel Base. Column 1 describes the ingredients as they are used in the formulation. Column 2 describes the final composition resulting from the in-situ formation of a total of 13.8% of niacinamide—organic acid complexes.

| Ingredient | Column 1 | Column 2 |
|---|---|---|
| Glycerin | 47.47 | 47.47 |
| Niacinamide | 10.0 | 3.9 |
| Water | 20.0 | 20.0 |
| Preservative | 0.5 | 0.5 |
| Dow Corning 2501 | 10.0 | 10.0 |
| Structure Plus | 4.0 | 4.0 |
| Eyebright Ext. | 0.1 | 0.1 |
| Botanical Ext. | 0.1 | 0.1 |
| Vitamin E | 0.1 | 0.1 |
| Lactic Acid | 0.9 | 0.0 |
| N-Acetyl-Cysteine | 1.63 | 0.0 |
| Ascorbic Acid | 1.76 | 0.0 |
| Salicylic Acid | 1.38 | 0.0 |
| Lipoic Acid | 2.06 | 0.0 |
| Niacinamide Lactate | 0.0 | 2.12 |
| Niacinamide Cysteinate | 0.0 | 2.85 |
| Niacinamide Ascorbate | 0.0 | 2.98 |
| Niacinamide Lipoate | 0.0 | 3.28 |
| Niacinamide Salicylate | 0.0 | 2.6 |

Procedure: All the ingredients in column 1 were mixed and heated at 60 to 70 C. for 30 minutes. A yellow mixture was obtained. The composition of this mixture is shown in Column 2. The product is Column 2 was obtained as a light yellow gel, useful for face and neck zone anti-acne, anti-age and anti-wrinkle applications. The product has the following properties: pH 4.5.

### Example 10

In-Situ Preparation of Niacinamide ascorbate and Niacinamide lactate with Hydroquinone. Column 1 shows the ingredients as they are added in the formulation. Column 2 shows the final composition of the formulation.

| Ingredient | Column 1 | Column 2 |
|---|---|---|
| PEG-6 | 54.66 | 54.66 |
| Aristoflex AVC | 0.8 | 0.8 |
| Water | 15.0 | 15.0 |
| Ascorbic Acid | 6.0 | 0.0 |
| Niacinamide | 6.44 | 0.0 |
| Hydroquinone | 4.0 | 4.0 |
| Silicone HPIB | 10.0 | 10.0 |
| Preservative | 0.3 | 0.3 |
| Lactic Acid | 1.8 | 0.0 |
| Niacinamide Ascorbate | 0.0 | 10.0 |
| Niacinamide lactate | 0.0 | 4.24 |

Process: Mix 2 and 3 till a clear gel is formed. Add 1, 7, and 8 and heat at 50 to 60 C. Add all other ingredients with mixing. Cool to room temperature. A translucent cream is obtained.

### Example 11

In-situ preparation of Benzyl niacin ascorbate, benzyl niacin lactate, and benzyl niacin lipoate. Column 1 shows the ingredients as they are added. Column 2 shows the final composition.

| Ingredient | Column 1 | Column 2 |
|---|---|---|
| PEG-6 | 50.29 | 50.29 |
| Aristoflex AVC | 1.0 | 1.0 |
| Glycerin | 5.0 | 5.0 |
| Water | 20.0 | 20.0 |
| Vitamin E | 2.1 | 2.1 |
| Preservative | 0.5 | 0.5 |
| Dimethicone | 4.0 | 4.0 |
| Benzyl Niacin | 6.39 | 0.0 |
| Ascorbic Acid | 1.76 | 0.0 |
| Lactic Acid | 0.9 | 0.0 |
| Lipoic Acid | 2.06 | 0.0 |
| Dimethiconol | 4.0 | 4.0 |
| Cetyl Dimethicone | 2.0 | 2.0 |
| Benzyl Niacin Ascorbate | 0.0 | 3.98 |
| Benzyl Niacin Lactate | 0.0 | 3.03 |
| Benzyl Niacin Lipoate | 0.0 | 4.1 |

Process. Mix 2, 3, and 4 till a clear gel is formed. Add all other ingredients and mix. A translucent cream is obtained. It is rapidly absorbed upon application to skin.

### Example 12

In-Situ Preparation of an Anhydrous 45.4% Methyl Niacin Lactate. Column 1 shows the ingredients as they are added. Column 2 shows the final composition.

| Ingredient | Column1 | Column 2 |
|---|---|---|
| PEG-6 | 54.6 | 54.6 |
| Methyl Niacin | 27.4 | 0.0 |
| Lactic Acid | 18.0 | 0.0 |
| Methyl Niacin Lactate | 0.0 | 45.4 |

Process: Mix all ingredients till a clear solution is obtained. The pH of this product is not important, as it does not contain any water (anhydrous composition).

### Example 13

Skin Penetration of Methyl Niacin, Lactic Acid, and In-Situ Generated Methyl Niacin Lactate Ion-Pair Complex.

| Ingredient | % Penetration |
|---|---|
| Methyl Niacin | 30.0 |
| Lactic Acid | 20.0 |
| Methyl Niacin Lactate | 81.0 |

Procedure. A 0.1 molar solution of ingredients in PEG-6 was applied on a synthetic membrane, which was placed over a Franz Diffusion Cell. The ingredients migrating to the phosphate buffer part of diffusion cell were quantified. % Penetration is based on the amount of ingredient applied to synthetic membrane.

### Example 14

In-Situ Preparation of Niacinamide salicylate and Niacinamide lipoate. Column 1 shows the ingredients as they are added. Column 2 shows the final composition.

| Ingredient | Column 1 | Column 2 |
|---|---|---|
| PEG-6 | 65.42 | 65.42 |
| Aristoflex AVC | 1.0 | 1.0 |
| Water | 15.1 | 15.1 |
| Salicylic Acid | 3.38 | 2.0 |
| Lipoic Acid | 2.06 | 0.0 |
| Niacinamide | 2.44 | 0.0 |
| Preservative | 0.3 | 0.3 |
| Polydimethylsiloxane | 10.0 | 10.0 |
| Botanical Ext. | 0.1 | 0.1 |
| Titanium dioxide | 0.2 | 0.2 |
| Niacinamide Salicylate | 0.0 | 2.6 |
| Niacinamide Lipoate | 0.0 | 3.28 |

Procedure: Mix all ingredients. Heat at 50 to 60 C. for 30 minutes till all solids dissolved. Cool to room temperature with slow mixing. A white cream is obtained.

### Example 15

A Method for Topical Application of 31.2% Niacinamide Hydroxycitrate Ion-Pair Complex. Method. The following are mixed together. (1) PEG-6 30.0, (2) Deionized Water 38.8 (3), Hydroxycitric acid 1 9.0, (4) Niacinamide 12.2. The resulting solution is applied to skin. A rapid absorption was noted.

### BRIEF DESCRIPTION OF THE DRAWINGS

FIG. 1. Various Five-Member Nitrogen Heterocyclic Bases.

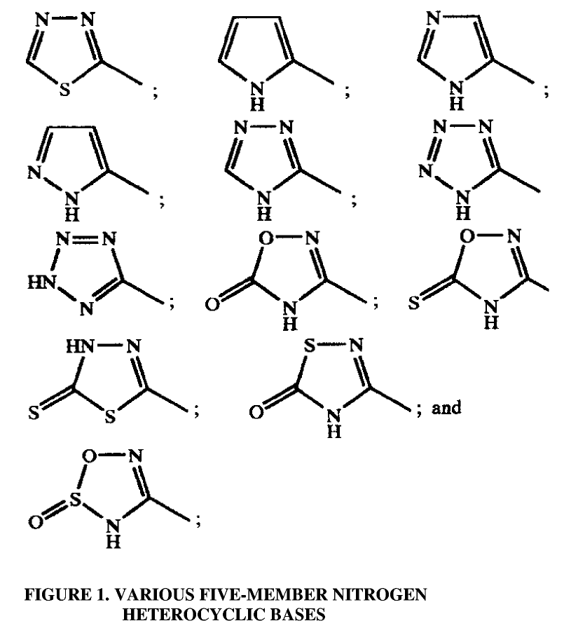

FIG. 2. % Skin Penetration of Organic Base and Base Salt Ingredients.

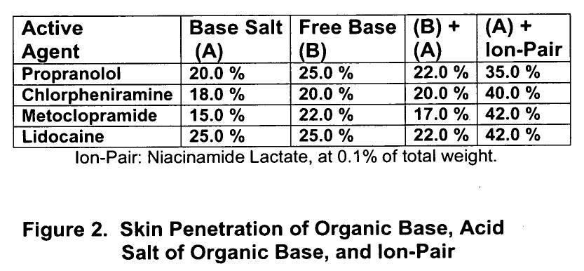

FIG. 3. pH Profile of Organic Base and Hydroxy Acid Active Agents and Their Ion-Pair Complexes.

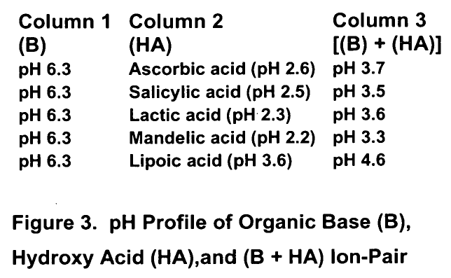

What is claimed is:

1. A method for concurrent enhancement of topical penetration of both an organic nitrogen heterocyclic base active agent and an organic hydroxy acid active agent, and comprising; (i) the organic nitrogen heterocyclic base active agent and organic hydroxy acid active agent are first combined in their equimolar weight percent amount to form an ion-pair complex, and (ii) said ion-pair complex is applied topically, whereupon said complex undergoes enhanced skin penetration, and (iii) upon reaching uppermost living part of skin and having reached therein the physiological pH of 7.4, said ion-pair complex dissociates and releases original constituent organic nitrogen heterocyclic base and organic acid active agents.

2. A method according to claim 1, wherein a solubilizing agent is included.

3. A method according to claim 1, wherein said organic nitrogen heterocyclic base active agent is selected from niacinamide, niacin alkyl esters, pyridoxine, and thiamine.

4. A method according to claim 1, wherein said organic nitrogen heterocyclic base active agent is niacinamide.

5. A method according to claim 1, wherein said organic hydroxy acid active agent is selected from salicylic acid, lactic acid, glycolic acid, malic acid, mandelic acid, ascorbic acid, ascorbyl phosphoric acid, hydroxycitric acid, Garcinia acid, Garcinia lactone, hydroxytetronic acid, phytic acid, aleuritic acid, ellagic acid, rosmarinic acid, chlorogenic acid, petroselinic acid, and combinations thereof.

6. A method according to claim 1, wherein said organic hydroxy acid active agent is Salicylic Acid.

7. A method according to claim 1, wherein said organic hydroxy acid active agent is Phytic Acid.

8. A method according to claim 1, wherein said organic hydroxy acid active agent is Ascorbic Acid.

9. A method according to claim 1, wherein said organic hydroxy acid active agent is Hydroxycitric Acid.

10. A method according to claim 1, wherein a cosmetic base is included.

11. A method according to claim 2, wherein solubilizing agent is selected from water, alcohol, glycol, polyethylene glycol, glycol ether, and combinations of said solubilizing agents.

12. A method according to claim 10, wherein vitamins, hormones, minerals, plant extracts, skin whitening agents, anti-inflammatory agents, concentrates of plant extracts, emollients, moisturizers, skin protectants, humectants, silicones, skin soothing ingredients, sun screens, analgesics, anesthetics, colorants, and perfumes are included.

13. A method of topical application of an ion-pair complex of an organic hydroxy acid active agent with an organic nitrogen heterocyclic base active agent, and comprising; (i) the in-situ preparation of said ion-pair complex of an organic hydroxy acid active agent with an organic nitrogen heterocyclic base active agent by mixing of said Hydroxy Acid and said organic nitrogen heterocyclic base in a 1:1 molar weight percent ratio, and wherein said organic nitrogen heterocyclic base having a pH of lower than 7.0 but higher than 3.0, when tested as a 1 percent solution or suspension in distilled water, and (ii) a solubilizing agent is included in 10 to 90 percent by weight, based the combined weight of said organic nitrogen heterocyclic base and said organic hydroxy acid, and (iii) mixing temperature of from 20 degrees Celsius to 90 degrees Celsius, and (iv) the ion-pair complex thus generated with a solubilizing agent is applied topically.

14. A method according to claim 13, wherein solubilizing agent is selected from water, alcohol, glycol, polyethylene glycol, glycol ether, and combinations of said solubilizing agents.

15. A method according to claim 13, wherein said ion-pair complex is Niacinamide Ascorbate.

16. A method of enhanced topical penetration of an ion-pair complex of an organic nitrogen heterocyclic base active agent and an organic hydroxy acid active agent upon upper, non-living layers of skin, and comprising; (i) said ion-pair complex is applied topically on upper, non-living layers of skin, and whereupon said ion-pair complex undergoes enhanced skin penetration as said ion-pair complex chemical entity without any dissociation, and (ii) upon reaching lower, living layers of skin, and having reached therein the physiological pH of 7.4, said ion-pair complex dissociates and releases its constituent organic nitrogen heterocyclic base and organic hydroxy acid active agent moieties.

17. A method according to claim 1 6, wherein a solubilizing agent is included, which is selected from water, alcohol, glycol, polyethylene glycol, glycol ether, and combinations of said solubilizing agents.

18. A method according to claim 16, wherein said ion-pair complex is selected from niacinamide lactate, niacinamide glycolate, niacinamide malate, niacinamide mandelate, niacinamide ascorbate, niacinamide phytate, niacinamide citrate, niacinamide hydroxy citrate, niacinamide aleurate, niacinamide salicylate, niacinamide hyaluronate, pyridoxine lactate, pyridoxine glycolate, pyridoxine malate, pyridoxine mandelate, pyridoxine ascorbate, pyridoxine phytate, pyridoxine citrate, pyridoxine hydroxycitrate, pyridoxine aleurate, pyridoxine salicylate, pyridoxine hyaluronate, and combinations thereof.

19. A method according to claim 16, wherein said ion-pair complex is Pyridoxine Salicylate.

20. A method according to claim 16, wherein a cosmetic base is included.
